(19) United States
(12) Patent Application Publication
(10) Pub. No.: US 2022/0330570 A1
(43) Pub. Date: Oct. 20, 2022

(54) METHOD AND SYSTEM FOR MANUFACTURING CHILL POPS

(71) Applicant: Shannon Miller, Milford, CA (US)

(72) Inventor: Shannon Miller, Milford, CA (US)

(21) Appl. No.: 17/231,378

(22) Filed: Apr. 15, 2021

(51) Int. Cl. A23G 9/36; A23G 9/08; A23G 9/42; A23L 33/105; B65D 85/78; A61K 31/047; A61K 31/352; A61K 9/00

(52) U.S. Cl. CPC A23G 9/36; A23G 9/083; A23G 9/42; A23L 33/105; B65D 85/78; A61K 31/047; A61K 31/352; A61K 9/0056; A23V 2002/00

(57) ABSTRACT

A method for producing a consumable product having ingredients or supplements with enhanced bioavailability or absorption rate by the consumer's body. The consumable product is disposed within a squeezable plastic tube which can be refrigerated or frozen. One of the sealed ends of the tube is removed to access the consumable product therein.

### RELATED APPLICATIONS

This application claims the benefit of U.S. Provisional Application No. 62/962,417, filed on Jan. 17, 2020.

### Background of the Invention

The present invention is generally related to processes and systems for manufacturing chill pops. More particularly, the present invention is directed to a system and method for manufacturing chill pops which are healthier and are created such that ingredients thereof have increased bioavailability to the consumer.

Chill pops, sometimes referred to as icy pops, freeze-and-eat pops, or squeeze freezer pops, such as that offered under the trademark Icee Pop™, are well known. Such chill pops are typically comprised of water with a high content of sugars and syrups and artificial food coloring, which are disposed within a plastic tube and frozen. An end of the plastic tube can be removed, such as by tearing, to access and consume the contents thereof. The contents can be moved towards the upper end by pinching and squeezing the tube from the bottom towards the upper end. The contents are typically frozen so as to mimic a popsicle. While tasty and refreshing, particularly on warmer days, such chill pops are not healthy due to their high level of sugar and artificial colorings.

It is also known to use such tubular packaging for yogurt, which is typically offered under the trademark Go-Gurt™. While this product offers yogurt, it also contains a large amount of sugar, as well as artificial coloring and flavors.

The packaging for the aforementioned products is convenient to the consumer as it can be frozen or refrigerated and easily transported and later consumed. However, the contents of these products are intended more as a snack or treat, particularly for children.

There are many products offered to consumers having ingredients which are intended to provide health or medicinal benefits. However, the bases of these products are such so as to merely deliver the ingredients to the body. There is little or no consideration to the absorption rate of these ingredients into the body. There are instances where the bioavailability, or increased absorption rate, would be beneficial to the consumer.

Accordingly, there is a continuing need for consumable products containing healthy ingredients and/or supplements which are designed so as to increase the bioavailability or absorption rate by the consumer's body. There is also a continuing need for providing such consumable products in convenient packaging. The present invention fulfills these needs, and provides other related advantages.

### SUMMARY OF THE INVENTION

The present invention resides in a method and system for manufacturing chill pops. In accordance with the invention, the bioavailability or absorption rate of ingredients, such as health or medicinal supplements, of the chill pop are increased. Such ingredients are provided in a convenient packaging, namely, a chill pop.

The method of creating a chill pop in accordance with the present invention comprises creating a consumable water-based base having a liquid or gel consistency. In a particularly preferred embodiment, guar gum is added with water to thicken the base and give the base antifreeze characteristics.

A health or medicinal supplement is added into the base to create a mixture. The supplement may comprise nano-CBD (cannabidiol), nano-THC (tetrahydrocannabinol), CBD, THC, medium-chain triglycerides (MCT) oil, branched-chain amino acids (BCAA), coconut water powder, alkaline water, green tea extract, beta alanine, and/or long jack extract.

The bioavailability of the supplement is enhanced by breaking down the supplement into nano-particles. This may be done by adding a nano-emulsifier to the mixture.

At least a portion of the mixture is inserted into an enclosed elongated flexible tube package. A sheet of plastic is formed into the tube package. The flexible tube package has a length of between four inches and two feet and a diameter of less than two inches. The tube package containing the mixture is refrigerated or frozen for later consumption.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a flow chart depicting steps taken in manufacturing a chill pop, in accordance with the present invention;

FIG. 2 is a front perspective view of an exemplary machine for manufacturing a chill pop in accordance with the present invention;

FIG. 3 is an enlarged sectioned view of a portion of the machine of FIG. 2, illustrating a roll of plastic film to create a flexible tube package, in accordance with the present invention;

FIG. 4 is a perspective view illustrating the plastic film being formed into a flexible tube package, in accordance with the present invention;

FIG. 5 is a perspective view illustrating a feed nozzle of the machine, used in accordance with the present invention;

FIG. 6 is a perspective view illustrating the filling of a flexible tube package in accordance with the present invention; and

FIG. 7 is a top view of a flexible tube package containing mixture in accordance with the present invention.

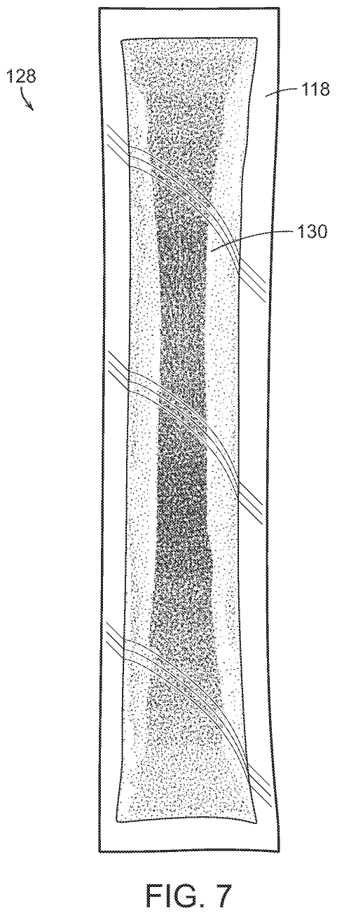

### DETAILED DESCRIPTION OF THE PREFERRED EMBODIMENTS

As shown in the accompanying drawings, for purposes of illustration, the present invention is directed to a method and system for manufacturing chill pops or the like having a consumable product with ingredients having increased bioavailability. Such ingredients may comprise health or medicinal supplements for the benefit and wellbeing of the user.

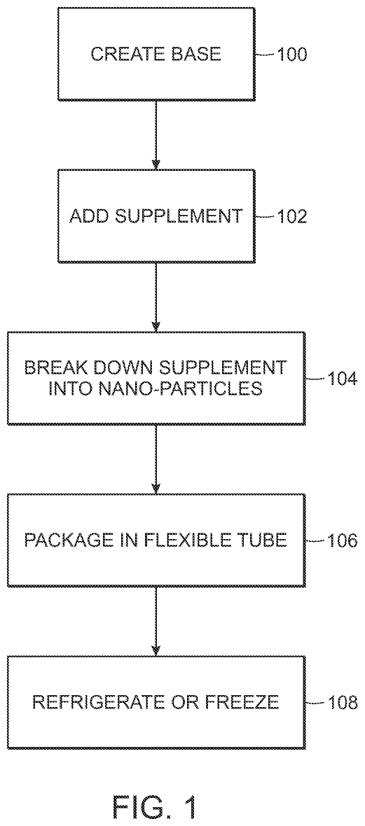

With reference now to FIG. 1, a flow chart is shown showing the general steps taken in manufacturing such chill pops in accordance with the present invention. A base is created 100. The base is consumable and preferably has a liquid or gel consistency. The base is comprised of water and a thickening agent. Whereas traditional chill pops utilize a high level of sugar content to create this consistency and provide antifreeze properties, in the present invention, in a particularly preferred embodiment, the thickening agent is guar gum.

Although sugars could be utilized in the product, preferably a no-calorie or low-calorie sweetener is used instead. Flavorings may be added, such as natural flavorings and/or sucralose or other artificial sweeteners or sugar substitutes.

In accordance with the present invention, one or more alternative ingredients, such as health or medicinal supplements, are also added to the base to create a mixture 102. These one or more ingredients may serve a particular purpose and have medicinal and/or health characteristics. Such ingredients may include nano-CBD, nano-THC, CBD, THC, MCT oil, BCAA, coconut water powder, alkaline water, green tea extract, beta alanine, and/or long jack extract. Such ingredients preferably impart a mental and/or health benefit to the consumer.

At least the supplement ingredients are broken down into nano-particles 104. In accordance with the present invention, the health or medicinal supplement ingredients are broken down into nano-particles to increase the bioavailability of the ingredients. This may be accomplished in a variety of manners. If the supplement or ingredient is available in an already “nano” form, this could be added to the mixture. Alternatively, the health or medicinal supplement and/or other ingredients may be subjected to a process, such as agitation or the like, which would break such supplements and other ingredients into nano-particles. In a particularly preferred embodiment of the invention, one or more nano-emulsifiers are added to the mixture to break down the health or medicinal supplements, and possibly other ingredients, into nano-particles. This creates a faster absorption rate by the body of these ingredients, which creates a more rapid delivery system to the body for these ingredients. This is particularly desirable when the ingredient is desired to be absorbed and utilized by the body more quickly, such as for sports applications, including post workout, medicinal or therapeutic purposes or the like. In accordance with the present invention, in a particularly preferred embodiment, the product is also healthy or dietary friendly so as to reduce or even eliminate the level of sugars and other unhealthy ingredients. Thus, a user could consume the product of the present invention without concern of gaining weight or other adverse health consequences.

At least a portion of the mixture is then inserted into a flexible tube package 106. The flexible tube package may be comprised of a sheet of plastic which is formed into a tube, with the mixture enclosed therein.

The flexible tube package having the mixture is then refrigerated, or more preferably frozen 108. As mentioned above, whereas traditional icee pops or other chill pops utilize a high level of sugar or syrup to aid in an antifreeze property of the product such that the product is partially frozen but more easily consumable, in the present invention guar gum, or other similar ingredient, serves to break down the ice in the final frozen product into smaller ice formations or otherwise impart an antifreeze property in addition to acting as a thickener for the base. In this manner, the partially frozen mixture is more easily consumed by the user.

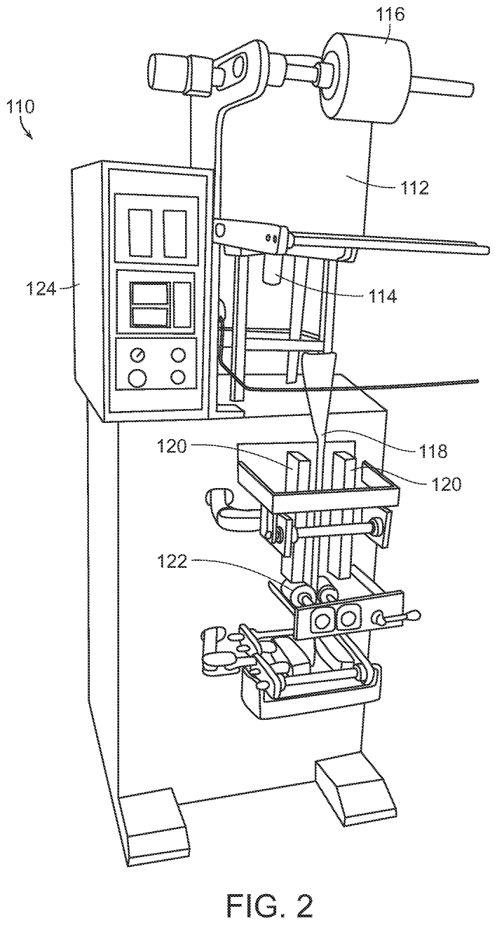

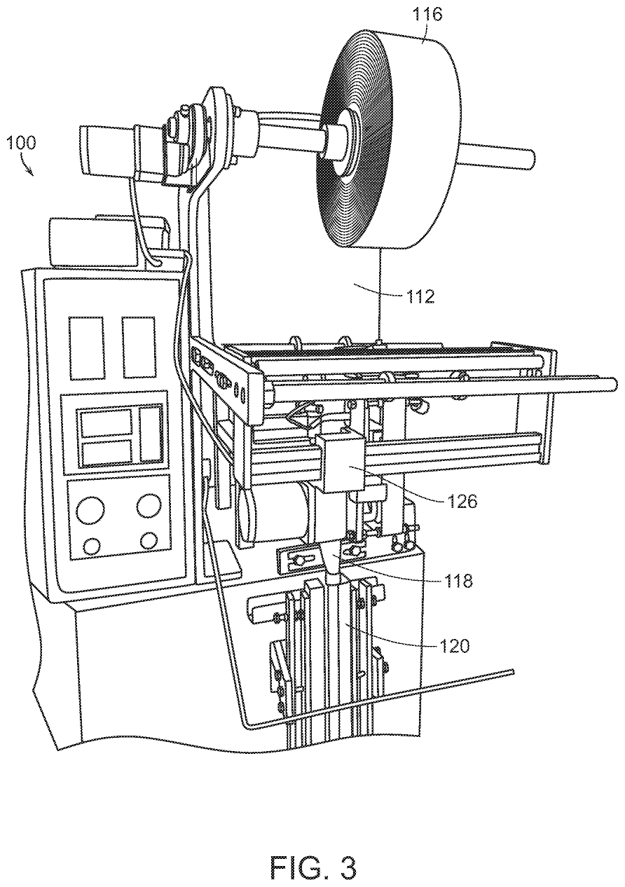

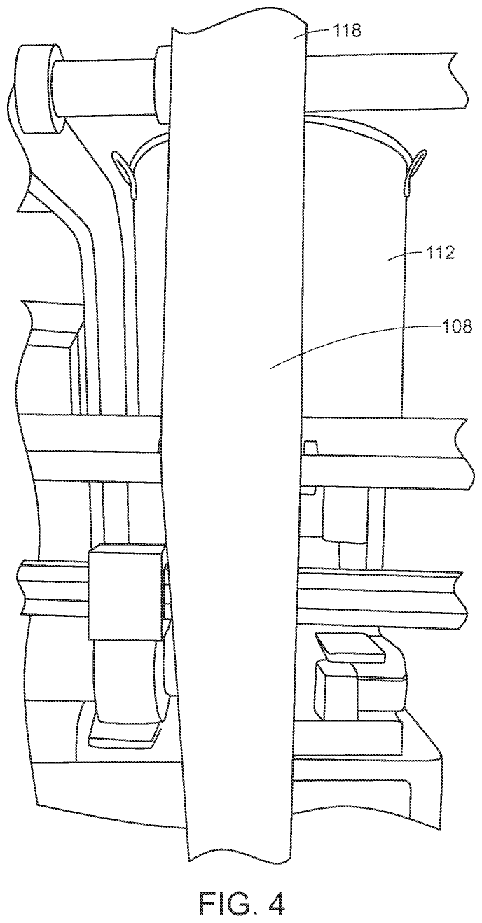

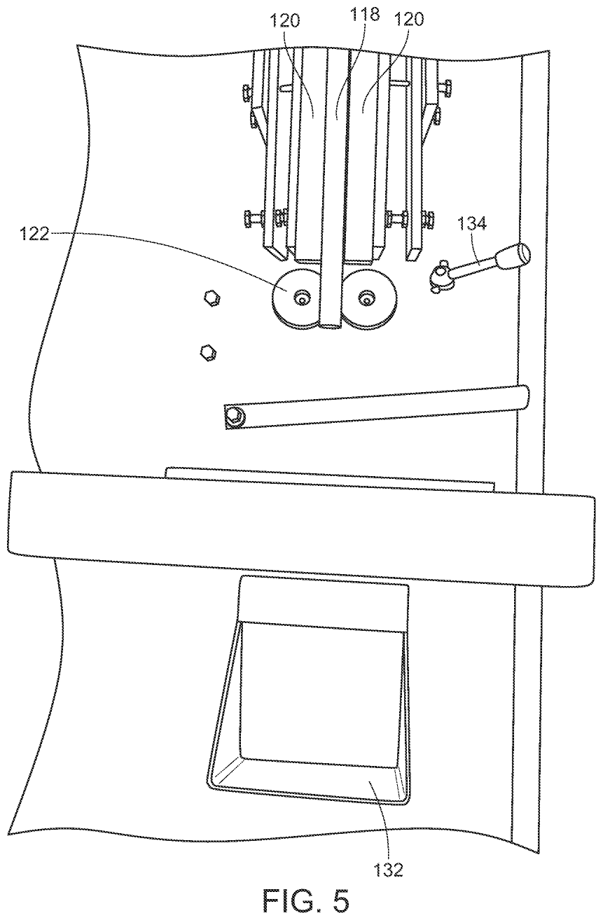

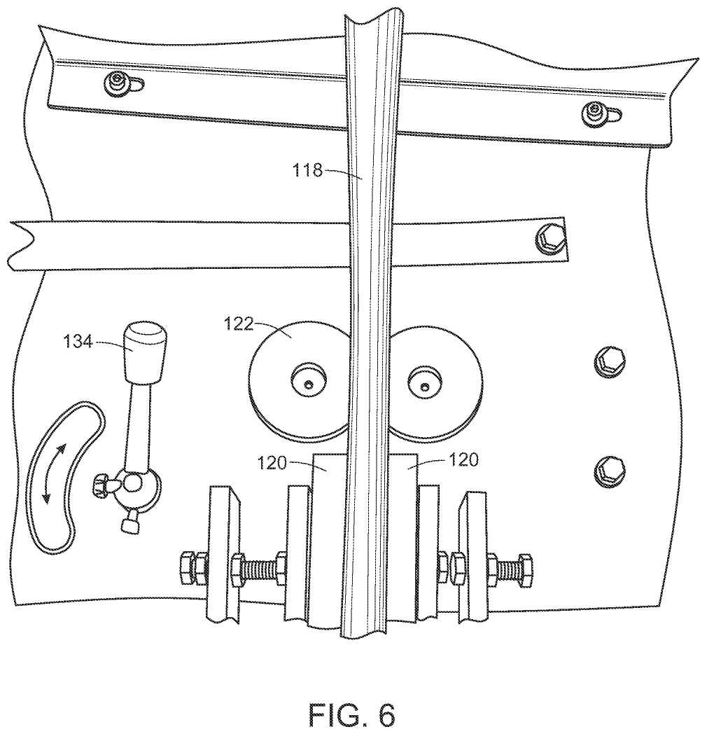

With reference now to FIGS. 2-6, after a mixture of the product has been made, it is then packaged in accordance with the present invention using a device 110, such as that illustrated in FIGS. 2-6. It will be appreciated that the illustrated device 110 is exemplary, and other devices and systems which package the mixture into enclosed flexible tube packages may be used instead. Such a device or system will insert at least a portion of the mixture into an enclosed elongated flexible tube package. Devices which create multiple packages simultaneously are also contemplated by the invention.

A batch of the mixture is poured into a top tank 112. A dispensing tube 114 extends from the tank 112 for transporting and delivering the liquid mixture. Thus, the tank 112 and the liquid mixture is in fluid communication with what will become the flexible tube packaging.

A spool of plastic sheet 116 is controllably fed by the device 110 such that the plastic sheet is folded or otherwise configured to create an elongated tube 118. Opposing edges of the plastic sheet may be heat sealed or otherwise attached to one another in order to create the flexible tube. For example, one or more heat sealers may be used to push together the back side of the packaging and seal it together and push together and heat seal an area of the tubular plastic sheet to create a bottom end of the product packaging. The plastic sheet 116 may have information printed thereon relating to the product, such as flavor, active or important ingredients and the like. At least one set of rollers 122 may be used to advance the flexible tube packaging, and guides 120 may also be used to control the advancement of the flexible tubing 118 through the device. The plastic sheet may include indicia readable or detectable by a sensor or scanner of the device 100 to assist in controllably feeding the plastic sheet and to create final product of a desired size.

A predetermined amount or portion of the mixture is inserted into the elongated flexible tube package 118. An electronic controller 124 may be used to coordinate the drawing of the plastic sheet from the spool 116, with the operation of the rollers 122 and guides 120 such that the flexible tube packaging is created and advanced and the liquid mixture inserted in an appropriate and desired amount. One or more sensors, such as an optical sensor 126, may read the plastic sheet roll 116 in order to ascertain and determine how far the roll of plastic sheet 116 is advanced before the heat sealer seals the backside packaging to seal it into a tube packaging, as well as a bottom end thereof before inserting the liquid mixture therein, and then heat sealing and cutting the flexible tube package once the desired amount of mixture is added thereto.

The result is an elongated flexible tube package 128 having the consumable mixture 130 disposed within the elongated plastic sheet tube, which is sealed at its ends to contain the mixture 130 therein. As the filled flexible tube packages are created, they are dispensed in a chute 132, or along a conveyor device or the like.

Although the electronic control box 124 enables the system operator to activate and deactivate the system and thus, in an automated fashion, feed the plastic sheet from the spool 116 to create the flexible tube, and dispense the liquid mixture into the tube, by controlling a variety of system components including motors, guides 120, rollers 122, sensors 126 and the like, heat sealers and cutters or the like, the device and system may also have a manual control 134, such as for manually overriding the control device 124 to cease operation. The lever switch 134 may also be used to selectively activate the operation of the device 110.

The process of forming the flexible tubes 118 from the sheet of spooled plastic 116, and subsequently filling the tubes with an appropriate amount of mixture, and sealing and cutting the flexible tube packaging 128 is repeated until the tank 112 is exhausted of mixture or the desired number of packages 128 have been produced.

The resultant filled tubular plastic packaging 128, containing the mixture with nano-emulsified ingredients of the invention, can then be chilled in a refrigerator or preferably partially frozen for later consumption. One sealed end of the package 128 can be removed, either by cutting or tearing the end, providing access to the consumable product 120 within the tubular plastic packaging 128. The user can then place this open end into his or her mouth to consume the product. The product may be advanced into the user's mouth by pinching or squeezing the opposite end of the tubular packaging 118 and advancing the product 130 upward towards the open end. Due to the use of the one or more nano-emulsifiers and/or the breakdown of ingredient particles into nano-particles, the active ingredients or ingredients of interest will be more rapidly delivered to the body and absorbed thereby.

Although several embodiments have been described in detail for purposes of illustration, various modifications may be made without departing from the scope and spirit of the invention. Accordingly, the invention is not to be limited, except as by the appended claims.

What is claimed is:

1. A method for creating a chill pop, comprising the steps of:
creating a consumable water-based base having a liquid or gel consistency;
adding a health or medicinal supplement into the base to create a mixture;
enhancing the bioavailability of the supplement by breaking down the supplement into nano-particles; and
inserting at least a portion of the mixture into an enclosed elongated flexible tube package.

2. The method of claim 1, including the step of refrigerating or freezing the package containing the mixture.

3. The method of claim 1, wherein the creating step includes the step of adding guar gum with the water to thicken the base and give the base antifreeze characteristics.

4. The method of claim 1, wherein the enhancing step comprises the step of adding a nano-emulsifier to the mixture.

5. The method of claim 1, wherein the supplement comprises nano-CBD, nano-THC, CBD, THC, MCT oil, BCAA, coconut water powder, alkaline water, green tea extract, beta alanine, and/or long jack extract.

6. The method of claim 1, including the step of forming a sheet of plastic into the tube package.

7. The method of claim 1, wherein the flexible tube package has a length of between four inches and two feet and a diameter of less than two inches.

8. A method for creating a chill pop, comprising the steps of:
creating a consumable base having a liquid or gel consistency, the base comprised of mixing guar gum with water to thicken the base and give the base antifreeze characteristics;
adding a health or medicinal supplement into the base to create a mixture;
adding a nano-emulsifier to the mixture to break down the supplement into nano-particles and enhance the bioavailability of the supplement; and
inserting at least a portion of the mixture into an enclosed elongated flexible tube package.

9. The method of claim 8, wherein the supplement comprises nano-CBD, nano-THC, CBD, THC, MCT oil, BCAA, coconut water powder, alkaline water, green tea extract, beta alanine, and/or long jack extract.

10. The method of claim 9, including the step of refrigerating or freezing the package containing the mixture.

11. The method of claim 8, including the step of forming a sheet of plastic into the tube package.

12. The method of claim 8, wherein the flexible tube package has a length of between four inches and two feet and a diameter of less than two inches.

13. A method for creating a chill pop, comprising the steps of:
creating a consumable base having a liquid or gel consistency, the base comprised of mixing guar gum with water to thicken the base and give the base antifreeze characteristics;
adding a health or medicinal supplement into the base to create a mixture, wherein the supplement comprises nano-CBD, nano-THC, CBD, THC, MCT oil, BCAA, coconut water powder, alkaline water, green tea extract, beta alanine, and/or long jack extract;
adding a nano-emulsifier to the mixture to break down the supplement into nano-particles and enhance the bioavailability of the supplement; and
forming a sheet of plastic into an elongated flexible tube package having a length of between four inches and two feet and a diameter of less than two inches;
inserting at least a portion of the mixture into the elongated flexible tube package; and
refrigerating or freezing the package containing the mixture.
